(19) United States
(12) Patent Application Publication
(10) Pub. No.: US 2020/0337675 A1
(43) Pub. Date: Oct. 29, 2020

(54) ULTRASOUND PROBE

(71) Applicant: OLYMPUS CORPORATION, Tokyo (JP)

(72) Inventor: Sunao SATO, Yamato-shi (JP)

(73) Assignee: OLYMPUS CORPORATION, Tokyo (JP)

(21) Appl. No.: 16/928,217

(22) Filed: Jul. 14, 2020

Related U.S. Application Data
(63) Continuation of application No. PCT/JP2018/046207, filed on Dec. 14, 2018.

(30) Foreign Application Priority Data
Jan. 16, 2018 (JP) 2018-005197

(51) Int. Cl. A61B 8/00

(52) U.S. Cl. CPC A61B 8/445; A61B 8/4281; A61B 8/4444

(57) ABSTRACT

An ultrasound probe includes: an ultrasound transmitting and receiving portion configured to transmit and receive ultrasound; and an acoustic lens configured to radiate ultrasound emitted from the ultrasound transmitting and receiving portion to outside and transmit ultrasound incident from outside to the ultrasound transmitting and receiving portion. The ultrasound transmitting and receiving portion and the acoustic lens each include a joint surface and a water repellent portion having water repellency disposed on an outer edge side of the joint surface, the joint surface of the ultrasound transmitting and receiving portion and the joint surface of the acoustic lens being joined to each other with an adhesive including a base resin and a curing agent.

### CROSS-REFERENCE TO RELATED APPLICATION

This application is a continuation of PCT International Application No. PCT/JP2018/046207 filed on Dec. 14, 2018, which designates the United States, incorporated herein by reference, and which claims the benefit of priority from Japanese Patent Application No. 2018-005197, filed on Jan. 16, 2018, incorporated herein by reference.

### BACKGROUND

### 1. Technical Field

The present disclosure relates to an ultrasound probe.

### 2. Related Art

In the related art, a ultrasound endoscope observes an inside of a subject, such as a human, using an ultrasound probe which is disposed on the distal end of a soft and elongated insertion unit by inserting the insertion unit into the subject (e.g., JP 5984525 B2).

The ultrasound probe described in JP 5984525 B2 includes an ultrasound transmitting and receiving portion that transmits and receives ultrasound and an acoustic lens that radiates ultrasound emitted from the ultrasound transmitting and receiving portion. The ultrasound transmitting and receiving portion and the acoustic lens are joined to each other with an adhesive.

### SUMMARY

In some embodiments, an ultrasound probe includes: an ultrasound transmitting and receiving portion configured to transmit and receive ultrasound; and an acoustic lens configured to radiate ultrasound emitted from the ultrasound transmitting and receiving portion to outside and transmit ultrasound incident from outside to the ultrasound transmitting and receiving portion. The ultrasound transmitting and receiving portion and the acoustic lens each include a joint surface and a water repellent portion having water repellency disposed on an outer edge side of the joint surface, the joint surface of the ultrasound transmitting and receiving portion and the joint surface of the acoustic lens being joined to each other with an adhesive including a base resin and a curing agent.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to a first embodiment;

FIG. 2 is a perspective view of the distal end of an insertion unit;

FIG. 3 is a sectional view of an ultrasound probe;

FIG. 4 is a diagram illustrating a fixing structure between an acoustic lens and an ultrasound transmitting and receiving portion with an adhesive;

FIG. 5 is a diagram illustrating the fixing structure between the acoustic lens and the ultrasound transmitting and receiving portion with the adhesive;

FIG. 6A is a diagram describing a method for fixing the acoustic lens and the ultrasound transmitting and receiving portion with the adhesive;

FIG. 6B is a diagram describing the method for fixing the acoustic lens and the ultrasound transmitting and receiving portion with the adhesive;

FIG. 7 is a diagram illustrating a fixing structure between an acoustic lens and an ultrasound transmitting and receiving portion with an adhesive according to a second embodiment;

FIG. 8 is a diagram illustrating the fixing structure between the acoustic lens and the ultrasound transmitting and receiving portion with the adhesive according to the second embodiment;

FIG. 9A is a diagram describing a problem in a conventional ultrasound probe; and

FIG. 9B is a diagram describing the problem in the conventional ultrasound probe.

### DETAILED DESCRIPTION

Hereinbelow, modes for carrying out the disclosure (hereinbelow, referred to as embodiments) will be described with reference to the drawings. Note that the disclosure is not limited to the embodiments described below. Further, identical reference signs designate identical elements throughout the drawings.

### First Embodiment

Schematic Configuration of Endoscope System

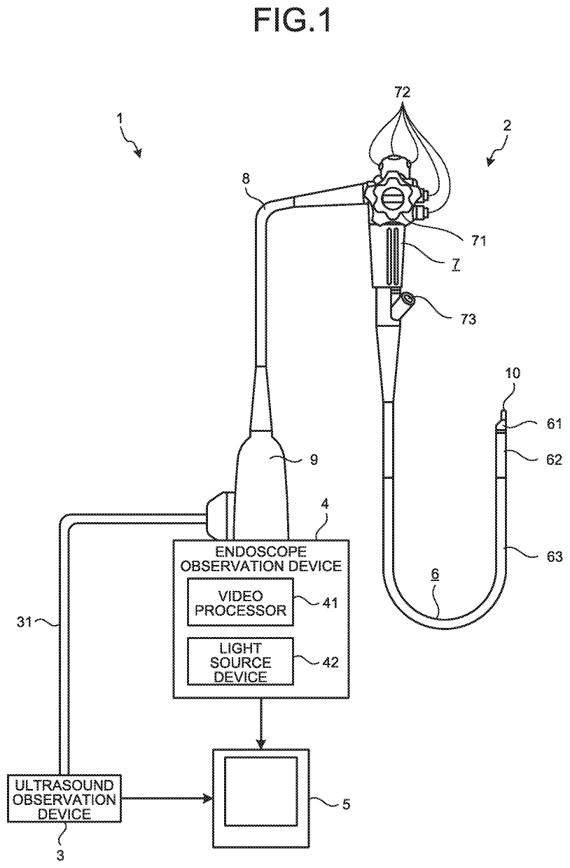

FIG. 1 is a diagram illustrating an endoscope system 1 according to a first embodiment.

The endoscope system 1 is a system that performs an ultrasound diagnosis on the inside of a subject, such as a human, using an ultrasound endoscope. As illustrated in FIG. 1, the endoscope system 1 includes an ultrasound endoscope 2, an ultrasound observation device 3, an endoscope observation device 4, and a display device 5.

The ultrasound endoscope 2 is partially insertable into a subject. The ultrasound endoscope 2 has a function of transmitting an ultrasound pulse (acoustic pulse) toward a body wall inside the subject, receiving an ultrasound echo reflected by the subject, and outputting an echo signal, and a function of capturing an image of the inside of the subject and outputting an image signal.

The detailed configuration of the ultrasound endoscope 2 will be described below.

The ultrasound observation device 3 is electrically connected to the ultrasound endoscope 2 through an ultrasound cable 31 (FIG. 1), and outputs a pulse signal to the ultrasound endoscope 2 and inputs an echo signal from the ultrasound endoscope 2 through the ultrasound cable 31. The ultrasound observation device 3 performs a predetermined process on the echo signal to generate an ultrasound image.

An endoscope connector 9 (FIG. 1, described below) of the ultrasound endoscope 2 is detachably connected to the endoscope observation device 4. As illustrated in FIG. 1, the endoscope observation device 4 includes a video processor 41 and a light source device 42.

The video processor 41 inputs an image signal from the ultrasound endoscope 2 through the endoscope connector 9. The video processor 41 performs a predetermined process on the image signal to generate an endoscope image.

The light source device 42 supplies illumination light which illuminates the inside of the subject to the ultrasound endoscope 2 through the endoscope connector 9.

The display device 5 includes liquid crystal or organic electro luminescence (EL). The display device 5 displays, for example, the ultrasound image generated by the ultrasound observation device 3 and the endoscope image generated by the endoscope observation device 4.

Configuration of Ultrasound Endoscope

Next, the configuration of the ultrasound endoscope 2 will be described.

As illustrated in FIG. 1, the ultrasound endoscope 2 includes an insertion unit 6, an operating unit 7, a universal cord 8, and the endoscope connector 9.

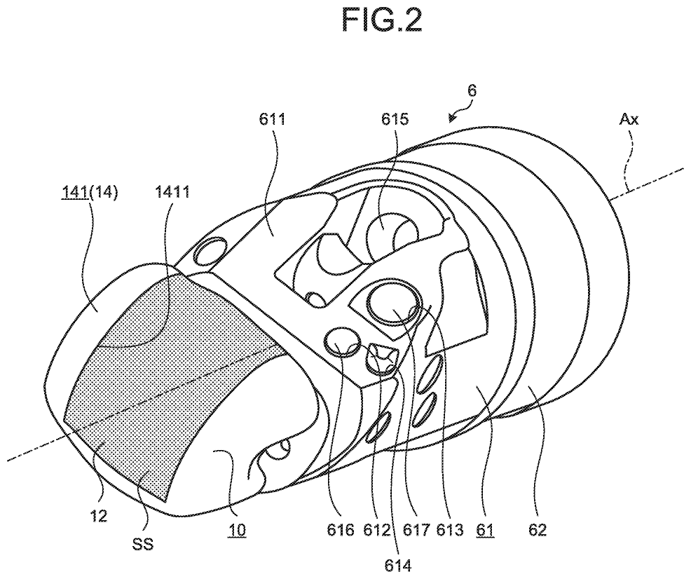

FIG. 2 is a perspective view of the distal end of the insertion unit 6.

Note that, in the following description for the configuration of the insertion unit 6, the distal end side of the insertion unit 6 (the distal end side in the insertion direction into the subject) is merely referred to as “distal end side”, and the proximal end side of the insertion unit 6 (the side opposite to the distal end of the insertion unit 6) is merely referred to as “proximal end side”.

The insertion unit 6 is a portion to be inserted into the subject. As illustrated in FIG. 1 or 2, the insertion unit 6 includes an ultrasound probe 10, which is disposed on the distal end side, a rigid member 61, which is coupled to the proximal end side of the ultrasound probe 10, a bendable portion 62, which is coupled to the proximal end side of the rigid member 61 and bendable, and a flexible tube 63, which is coupled to the proximal end side of the bendable portion 62 and has flexibility.

Inside the insertion unit 6, the operating unit 7, the universal cord 8, and the endoscope connector 9, a light guide (not illustrated) which transmits the illumination light supplied from the light source device 42, a transducer cable (not illustrated) which transmits the above-mentioned pulse signal and the above-mentioned echo signal, and a signal cable (not illustrated) which transmits the image signal are routed, and a duct (not illustrated) for circulating a fluid therethrough is disposed.

The rigid member 61 is a hard member including, for example, a resin material, and has a substantially columnar shape extending along an insertion axis Ax (FIG. 2). The insertion axis Ax is an axis extending in the extending direction of the insertion unit 6.

The rigid member 61 includes an inclined surface 611, which is formed on the outer peripheral face of the rigid member 61 at the distal end side to constitute a tapered shape of the rigid member 61 tapered toward the distal end.

As illustrated in FIG. 2, the rigid member 61 includes a mounting hole (not illustrated) which penetrates the rigid member 61 from the proximal end to the distal end thereof, and an illumination hole 612, an imaging hole 613, an air/water supply hole 614, and a treatment tool channel 615, each of which penetrates the rigid member 61 from the proximal end to the inclined surface 611 thereof.

The above-mentioned mounting hole (not illustrated) is a hole in which the ultrasound probe 10 is mounted. The above-mentioned transducer cable (not illustrated) is inserted inside the mounting hole.

The emitting end side of the above-mentioned light guide (not illustrated) and an illumination lens 616 (FIG. 2), which applies illumination light emitted from the emitting end of the light guide to the inside of the subject, are disposed inside the illumination hole 612.

An objective optical system 617 (FIG. 2), which condenses light applied to and reflected by the inside of the subject (subject image), and an image sensor (not illustrated) which captures the subject image condensed by the objective optical system 617 are disposed inside the imaging hole 613. The image signal captured by the image sensor is transmitted to the endoscope observation device 4 (the video processor 41) through the above-mentioned signal cable (not illustrated).

In the first embodiment, the illumination hole 612 and the imaging hole 613 are formed on the inclined surface 611 as described above. Thus, the ultrasound endoscope 2 according to the first embodiment is configured as an oblique viewing endoscope which performs an observation in a direction intersecting the insertion axis Ax at an acute angle.

The air/water supply hole 614 constitutes a part of the above-mentioned duct (not illustrated). The air/water supply hole 614 is a hole for supplying air or water toward the imaging hole 613 to clean the outer surface of the objective optical system 617.

The treatment tool channel 615 is a passage that allows a treatment tool (not illustrated), such as a puncture needle, inserted inside the insertion unit 6 to protrude to the outside.

The operating unit 7 is a portion that is coupled to the proximal end side of the insertion unit 6 and receives various operations by a medical doctor or the like. As illustrated in FIG. 1, the operating unit 7 includes a bending knob 71 for bending the bendable portion 62 and a plurality of operating members 72 for performing various operations.

Further, the operating unit 7 includes a treatment tool insertion port 73, which communicates with the treatment tool channel 615 through a tube (not illustrated) disposed inside the bendable portion 62 and the flexible tube 63 to insert the treatment tool (not illustrated) into the tube.

The universal cord 8 extends from the operating unit 7. The universal cord 8 is a cord in which the above-mentioned light guide (not illustrated), the above-mentioned transducer cable (not illustrated), the above-mentioned signal cable (not illustrated), and a tube (not illustrated) that constitutes a part of the above-mentioned duct (not illustrated) are disposed.

The endoscope connector 9 is disposed on an end of the universal cord 8. The ultrasound cable 31 is connected to the endoscope connector 9, and the endoscope connector 9 is inserted into the endoscope observation device 4 so as to be connected to the video processor 41 and the light source device 42.

Configuration of Ultrasound Probe

Next, the configuration of the ultrasound probe 10 will be described.

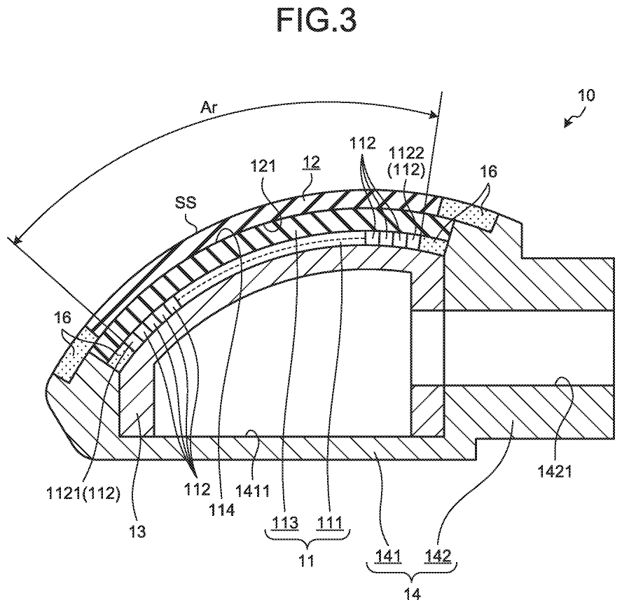

FIG. 3 is a sectional view of the ultrasound probe 10. Specifically, FIG. 3 is a sectional view of the ultrasound probe 10 taken along a plane including the insertion axis Ax and perpendicular to a scanning surface SS.

The ultrasound probe 10 is a convex ultrasound probe, and includes the scanning surface SS, which has a cylindrical surface shape convex outward (upward in FIG. 3),

In the following description for the configuration of the ultrasound probe 10, the circumferential direction on the scanning surface SS having the cylindrical surface shape is merely referred to as “circumferential direction”, and a direction along a cylindrical axis on the scanning surface SS having the cylindrical surface shape (the direction perpendicular to the sheet in FIG. 3) is referred to as “width direction”.

The ultrasound probe 10 performs a scan with ultrasound (transmits and receives ultrasound) in the circumferential direction within an ultrasound transmitting and receiving area Ar (FIG. 3), which is defined by normal lines of the scanning surface SS and has a fan shape in sectional view.

As illustrated in FIG. 3, the ultrasound probe 10 includes an ultrasound transmitting and receiving portion 11, an acoustic lens 12, a backing material 13, and a holding member 14.

The ultrasound transmitting and receiving portion 11 is a portion that transmits and receives ultrasound. As illustrated in FIG. 3, the ultrasound transmitting and receiving portion 11 includes a transducer 111, which includes a plurality of piezoelectric elements 112, and an acoustic matching layer 113.

Each of the piezoelectric elements 112 has a long rectangular parallelepiped shape linearly extending in the width direction. As illustrated in FIG. 3, the piezoelectric elements 112 are regularly arranged in the circumferential direction. Further, although not specifically illustrated, a pair of electrodes is formed on the outer surface of the piezoelectric element 112. The piezoelectric element 112 converts a pulse signal (corresponding to the electrical signal according to the disclosure) input through the above-mentioned transducer cable (not illustrated) and the pair of electrodes (not illustrated) to an ultrasound pulse, and transmits the ultrasound pulse to the subject. Further, the piezoelectric element 112 converts an ultrasound echo reflected by the subject to an electrical echo signal (corresponding to the electrical signal according to the disclosure) represented by a voltage change, and outputs the electrical echo signal to the transducer cable (not illustrated) through the above-mentioned pair of electrodes (not illustrated).

More specifically, the position of one end of the ultrasound transmitting and receiving area Ar in the circumferential direction corresponds to the position of a piezoelectric element 1121 (FIG. 3), which is located at one end in the circumferential direction among the piezoelectric elements 112. Further, the position of the other end of the ultrasound transmitting and receiving area Ar in the circumferential direction corresponds to the position of a piezoelectric element 1122 (FIG. 3), which is located at the other end in the circumferential direction among the piezoelectric elements 112.

The piezoelectric element 112 includes PMN-PT single crystal, PMN-PZT single crystal, PZN-PT single crystal, PIN-PZN-PT single crystal, or a relaxor-based material.

The PMN-PT single crystal is an abbreviation of a solid solution of lead magnesium niobate and lead titanate. The PMN-PZT single crystal is an abbreviation of a solid solution of lead magnesium niobate and lead zirconate titanate. The PZN-PT single crystal is an abbreviation of a solid solution of lead zinc niobate and lead titanate. The PIN-PZN-PT single crystal is an abbreviation of a solid solution of lead indium niobate, lead zinc niobate, and lead titanate. The relaxor-based material is a general term of a three-component piezoelectric material obtained by adding lead-based complex perovskite as a relaxor material to the lead zirconate titanate (PZT) for the purpose of increasing the piezoelectric constant and dielectric constant. The lead-based complex perovskite is represented by Pb(B1, B2)O₃, where B1 is any of magnesium, zinc, indium, and scandium, and B2 is any of niobium, tantalum, and tungsten. These materials have excellent piezoelectric effects. Thus, these materials can reduce the value of the electrical impedance even in a downsized form, and are, thus, preferred from the viewpoint of impedance matching with the above-mentioned pair of electrodes (not illustrated).

As illustrated in FIG. 3, the acoustic matching layer 113 extends in the circumferential direction, and is stacked on the transducer 111 at the side corresponding to the outer surface side of the ultrasound probe 10 (the upper side in FIG. 3). In order to efficiently transmit sound (ultrasound) between the transducer 111 (piezoelectric element 112) and the subject, the acoustic matching layer 113 performs acoustic impedance matching between the transducer 111 and the subject.

In the first embodiment, as illustrated in FIG. 3, the length dimension of the acoustic matching layer 113 in the circumferential direction is set larger than the length dimension of the transducer 111 in the circumferential direction.

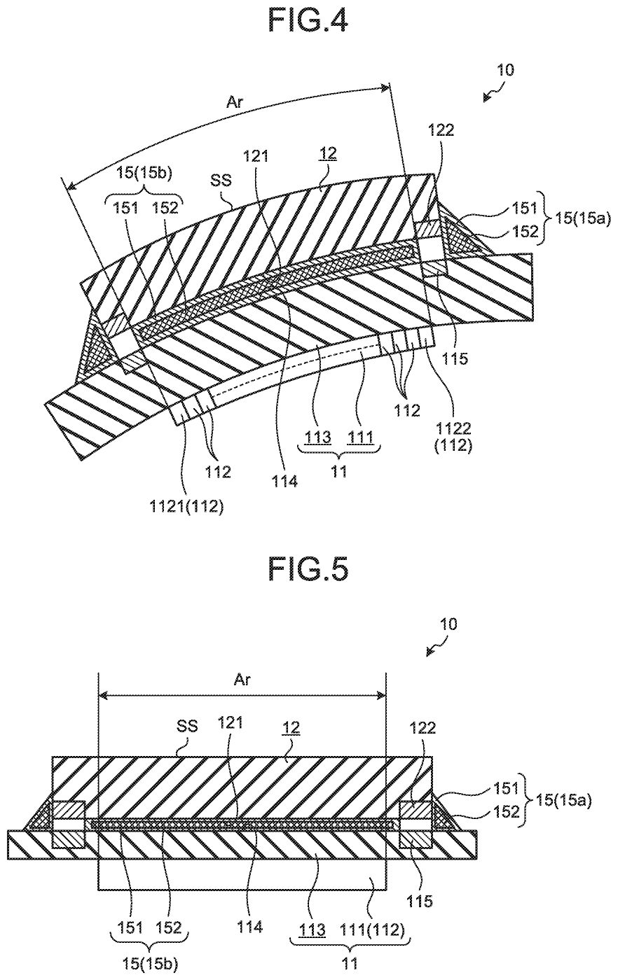

The acoustic lens 12 includes, for example, a silicone resin. As illustrated in FIG. 3, the acoustic lens 12 is a plate that extends in the circumferential direction and has an arc shape in sectional view. The acoustic lens 12 is fixed onto the ultrasound transmitting and receiving portion 11 (the acoustic matching layer 113) with an adhesive 15 (refer to FIGS. 4 and 5) at the side corresponding to the outer surface side of the ultrasound probe 10. In other words, one plate surface (the plate surface on the upper side in FIG. 3) of the acoustic lens 12 serves as the scanning surface SS. The acoustic lens 12 converges the ultrasound pulse transmitted from the transducer 111 through the acoustic matching layer 113. Further, the acoustic lens 12 transmits the ultrasound echo reflected by the subject to the acoustic matching layer 113.

In the first embodiment, as illustrated in FIG. 3, the length dimension of the acoustic lens 12 in the circumferential direction is set smaller than the length dimension of the acoustic matching layer 113 in the circumferential direction and larger than the length dimension of the transducer 111 in the circumferential direction.

The fixing structure between the acoustic lens 12 and the ultrasound transmitting and receiving portion 11 with the adhesive 15 will be described below.

As illustrated in FIG. 3, the backing material 13 is a member that is disposed with the transducer 111 interposed between the backing material 13 and the acoustic matching layer 113 to attenuate unnecessary ultrasound vibration generated by the operation of the piezoelectric element 112. The backing material 13 includes a material having a high attenuation factor, for example, an epoxy resin in which a filler such as alumina or zirconia is dispersed, or a rubber in which the above-mentioned filler is dispersed.

As illustrated in FIG. 3, the holding member 14 includes a holding portion 141 and an attachment portion 142.

The holding portion 141 is a portion that holds a unit including the ultrasound transmitting and receiving portion 11, the acoustic lens 12, and the backing material 13 which are integrated together. As illustrated in FIG. 3, the holding portion 141 includes a recess 1411, which exposes the scanning surface SS of the acoustic lens 12 to the outside while holding the unit. A gap between the recess 1411 and the unit is filled with an adhesive 16 (FIG. 3).

The attachment portion 142 is a portion that is formed integrally with the proximal end of the holding portion 141, and inserted into the above-mentioned mounting hole (not illustrated) of the rigid member 61 and attached to the rigid member 61. As illustrated in FIG. 3, the attachment portion 142 includes an insertion hole 1421, which penetrates the attachment portion 142 from the proximal end thereof to the recess 1411, and the above-mentioned transducer cable (not illustrated) is inserted into the insertion hole 1421.

Fixing Structure between Acoustic Lens and Ultrasound Transmitting and receiving portion with Adhesive

Next, the fixing structure between the acoustic lens 12 and the ultrasound transmitting and receiving portion 11 with the adhesive 15 will be described.

FIGS. 4 and 5 are diagrams illustrating the fixing structure between the acoustic lens 12 and the ultrasound transmitting and receiving portion 11 with the adhesive 15. Specifically, FIG. 4 is a sectional view of the acoustic lens 12 and the ultrasound transmitting and receiving portion 11 taken along a plane including the insertion axis Ax and perpendicular to the scanning surface SS. FIG. 5 is a sectional view of the acoustic lens 12 and the ultrasound transmitting and receiving portion 11 taken along a plane perpendicular to the scanning surface SS and extending in the width direction.

The adhesive 15 is a two-part adhesive, and includes a base resin 151 and a curing agent 152 as illustrated in FIGS. 4 and 5. For convenience of explanation, FIGS. 4 and 5 illustrate a separated state of the base resin 151 and the curing agent 152, which are mixed together in actuality.

As illustrated in FIGS. 4 and 5, the acoustic matching layer 113 includes a first water repellent portion 115 having water repellency, which is disposed on the outer edge side of a joint surface 114, which is jointed to the acoustic lens 12 with the adhesive 15.

On the other hand, as illustrated in FIGS. 4 and 5, the acoustic lens 12 includes a second water repellent portion 122 having water repellency, which is disposed on the outer edge side of a joint surface 121 (the plate surface opposite to the scanning surface SS), which is joined to the acoustic matching layer 113 with the adhesive 15, at the position facing the first water repellent portion 115.

The above-mentioned first and second water repellent portions 115 and 122 correspond to the water repellent portions according to the disclosure. In the first embodiment, the first and second water repellent portions 115 and 122 have frame shapes extending along the outer edges of the joint surfaces 114 and 121, respectively. As illustrated in FIGS. 4 and 5, the first and second water repellent portions 115 and 122 are disposed outside the ultrasound transmitting and receiving area Ar on the stacked cross-section of the acoustic lens 12 and the ultrasound transmitting and receiving portion 11. Further, each of the first and second water repellent portions 115 and 122 includes a water repellent coating film (fluorine coating film).

Each of the first and second water repellent portions 115 and 122 is not limited to the fluorine coating film, and may include a hydrophobic silica coating film, or may have a double roughness structure. Specifically, the double roughness structure is a structure (lotus leaf structure) having a plurality of micrometer-size projections (concave-convex shape) including nanometer-size projections.

In the configuration of the first and second water repellent portions 115 and 122, the water repellent coating film such as the fluorine coating film or the hydrophobic silica coating film is more easily manufactured at lower cost than the double roughness structure, while the double roughness structure has a higher water repellency than the water repellent coating film.

The “water repellency” in the first embodiment means, for example, a property that causes a water droplet to be in contact with a water droplet holding surface at a contact angle greater than 90°. Further, “super-water repellency” which is a property that causes a water droplet to be in contact with a water droplet holding surface with a contact angle greater than 150° is more preferred.

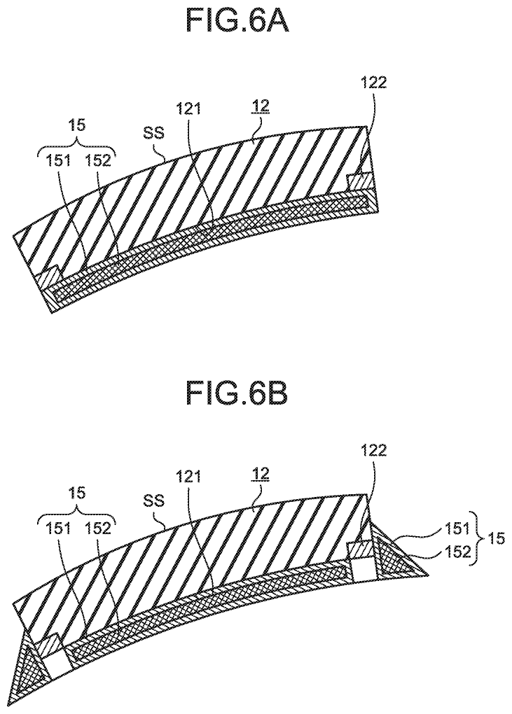

FIGS. 6A and 6B are diagrams describing a method for fixing the acoustic lens 12 and the ultrasound transmitting and receiving portion 11 with the adhesive 15. Specifically, FIGS. 6A and 6B correspond to FIG. 4. In a manner similar to FIG. 4, FIGS. 6A and 6B illustrate a separated state of the base resin 151 and the curing agent 152, which are mixed together in actuality.

First, as illustrated in FIG. 6A, an operator applies the adhesive 15 to the entire joint surface 121 of the acoustic lens 12.

As described above, the joint surface 121 includes the second water repellent portion 122. Thus, as illustrated in FIG. 6B, a part of the adhesive 15 applied to the entire joint surface 121, the part being applied to the second water repellent portion 122, moves into an area surrounded by the second water repellent portion 122 or moves out of the area due to the water repellency of the second water repellent portion 122.

Next, the operator puts the acoustic lens 12 illustrated in FIG. 6B onto the joint surface 114 of the acoustic matching layer 113 and allows the adhesive 15 to be cured to fix the acoustic lens 12 and the ultrasound transmitting and receiving portion 11 to each other.

The adhesive 15 may adhere to the first water repellent portion 115 of the joint surface 114 of the acoustic matching layer 113 when the acoustic lens 12 is put onto the joint surface 114. Even in such a case, the adhesive 15 adhering to the first water repellent portion 115 moves into an area surrounded by the first water repellent portion 115 or moves out of the area due to the water repellency of the first water repellent portion 115. In other words, as illustrated in FIG. 4 or 5, the adhesive 15 is not present between the first and second water repellent portions 115 and 122 in a state in which the acoustic lens 12 and the ultrasound transmitting and receiving portion 11 are fixed to each other.

The first embodiment described above achieves the following effects.

In the ultrasound probe 10 according to the first embodiment, the first and second water repellent portions 115 and 122 having water repellency are disposed on the outer edge side of the joint surface 114 of the ultrasound transmitting and receiving portion 11 and on the outer edge side of the joint surface 121 of the acoustic lens 12, respectively.

Thus, immediately after the adhesive 15 is applied, the adhesive 15 adhering to the first and second water repellent portions 115 and 122 moves into the areas surrounded by the first and second water repellent portions 115 and 122 or moves out of the areas. In other words, it is possible to prevent either the base resin 151 or the curing agent 152 having a lower viscosity from being drawn into the gap between the joint surfaces 114 and 121 due to a capillary phenomenon. As a result, it is possible to keep the balance of the combination ratio in the adhesive 15 both inside and outside the areas surrounded by the first and second water repellent portions 115 and 122, which enables the adhesive 15 to be appropriately cured.

Thus, it is possible to check that an adhesive 15b (FIGS. 4 and 5) present inside (inside the areas surrounded by the first and second water repellent portions 115 and 122) has been cured by checking that an adhesive 15a (FIGS. 4 and 5) present outside the areas surrounded by the first and second water repellent portions 115 and 122 has been cured.

Further, in the ultrasound probe 10 according to the first embodiment, the first and second water repellent portions 115 and 122 have frame shapes extending along the outer edges of the joint surfaces 114 and 121, respectively.

Thus, it is possible to appropriately cure the adhesive 15a on the entire outer edges. This eliminates the necessity of an operation for wiping off the adhesive 15a uncured on the outer edges, which simplifies the manufacturing operation.

Furthermore, in the ultrasound probe 10 according to the first embodiment, the first and second water repellent portions 115 and 122 are disposed outside the ultrasound transmitting and receiving area Ar on the stacked cross-section of the acoustic lens 12 and the ultrasound transmitting and receiving portion 11.

Thus, ultrasound is not affected by the first and second water repellent portions 115 and 122, which enables the generation of a satisfactory ultrasound image.

### Second Embodiment

Next, a second embodiment will be described.

In the following description, elements similar to those of the above first embodiment are designated by the same reference signs as the first embodiment, and detailed description thereof will be omitted or simplified.

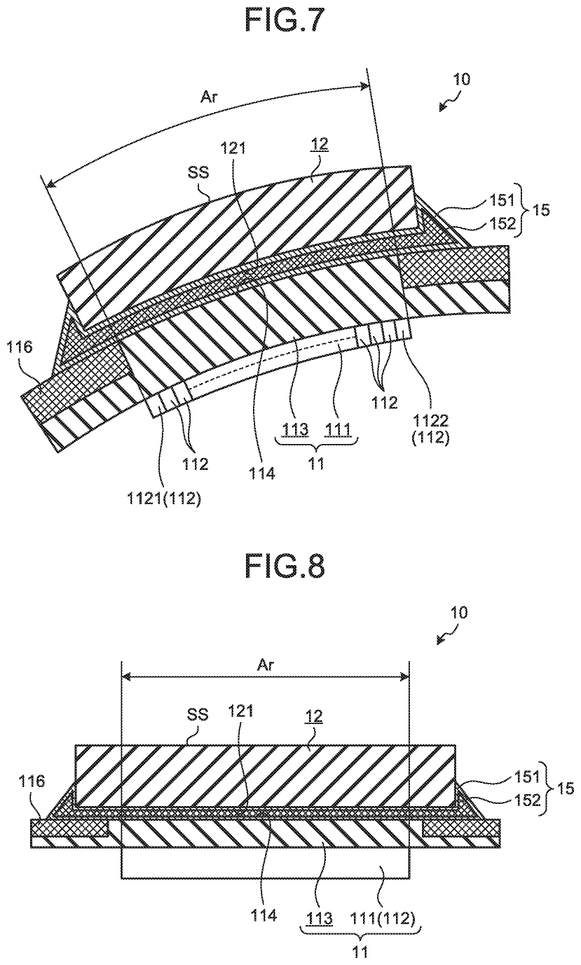

FIGS. 7 and 8 are diagrams illustrating a fixing structure between an acoustic lens 12 and an ultrasound transmitting and receiving portion 11 with an adhesive 15 according to the second embodiment. Specifically, FIGS. 7 and 8 correspond to FIGS. 4 and 5, respectively.

As illustrated in FIG. 7 or 8, the second embodiment differs from the first embodiment only in that the acoustic lens 12 does not include the second water repellent portion 122, and an acoustic matching layer 113 includes a heat absorbing structure 116, which absorbs heat of the adhesive 15, instead of the first water repellent portion 115.

As illustrated in FIG. 7 or 8, the heat absorbing structure 116 has a frame shape extending along the outer edge of a joint surface 114. In the second embodiment, the heat absorbing structure 116 is disposed outside the ultrasound transmitting and receiving area Ar on the stacked cross-section of the acoustic lens 12 and the ultrasound transmitting and receiving portion 11. The heat absorbing structure 116 includes, for example, a metal film, a conductive adhesive, or carbon.

The second embodiment described above achieves the following effects.

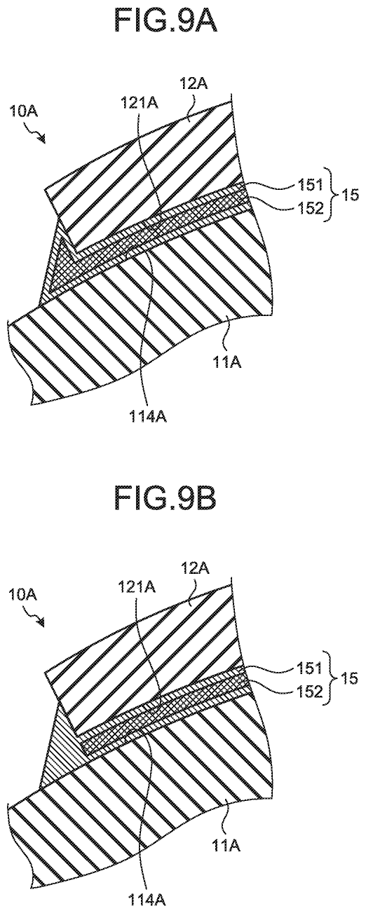

Incidentally, even in a case where there is no difference in viscosity between the base resin 151 and the curing agent 152 at ordinary temperatures, a temperature rise caused by self-heating during curing may produce a difference in viscosity between the base resin 151 and the curing agent 152. This causes a phenomenon that the adhesive 15 is not cured due to an imbalance in the combination ratio between the base resin 151 and the curing agent 152 on the outer edge side as described above with reference to FIG. 9B.

The ultrasound probe 10 according to the second embodiment includes the heat absorbing structure 116, which is disposed on the outer edge side of the joint surface 114 of the ultrasound transmitting and receiving portion 11.

Accordingly, heat of the adhesive 15 is absorbed by the heat absorbing structure 116. Thus, it is possible to reduce a temperature rise caused by self-heating during curing to avoid a difference in viscosity between the base resin 151 and the curing agent 152. Therefore, it is possible to prevent either the base resin 151 or the curing agent 152 having a lower viscosity from being drawn into the gap between the joint surfaces 114 and 121 due to a capillary phenomenon. As a result, it is possible to keep the balance of the combination ratio in the entire adhesive 15, which enables the adhesive 15 to be appropriately cured.

Thus, it is possible to check that the adhesive 15 present inside has been cured by checking that the adhesive 15 present on the outer edge side has been cured.

Further, in the ultrasound probe 10 according to the second embodiment, the heat absorbing structure 116 has a frame shape extending along the outer edge of the joint surface 114.

Thus, it is possible to appropriately cure the adhesive 15 on the entire outer edge. This eliminates the necessity of an operation for wiping off the adhesive 15 uncured on the outer edge, which simplifies the manufacturing operation.

Furthermore, in the ultrasound probe 10 according to the second embodiment, the heat absorbing structure 116 is disposed outside the ultrasound transmitting and receiving area Ar on the stacked cross-section of the acoustic lens 12 and the ultrasound transmitting and receiving portion 11.

Thus, ultrasound is not affected by the heat absorbing structure 116, which enables the generation of a satisfactory ultrasound image.

### Other Embodiments

The embodiments of the disclosure have been described above. However, the disclosure is not limited only to the above first and second embodiments.

Although, in the above first and second embodiments, the ultrasound probe 10 is configured as a convex ultrasound probe, the disclosure is not limited thereto. The ultrasound probe 10 may be configured as a radial ultrasound probe.

Although, in the above first and second embodiments, the endoscope system 1 has both the function of generating an ultrasound image and the function of generating the endoscope image, the disclosure is not limited thereto. The endoscope system 1 may have only the function of generating an ultrasound image.

In the above first and second embodiments, the endoscope system 1 may be not only an endoscope system used in the medical field, but also an endoscope system that observes the inside of a subject such as a mechanical structure in the industrial field.

Although, in the above first and second embodiments, the ultrasound endoscope 2 is configured as an oblique viewing endoscope which performs an observation in the direction intersecting the insertion axis Ax at an acute angle, the disclosure is not limited thereto. The ultrasound endoscope 2 may be a side viewing endoscope which performs an observation in a direction perpendicular to the insertion axis Ax or a forward viewing endoscope which performs an observation in a direction parallel to the insertion axis Ax.

In the above first and second embodiments, a configuration that does not include the acoustic matching layer 113 (the configuration in which the acoustic lens 12 is directly bonded and fixed to the transducer 111) may be employed. In this case, the transducer 111 may include the first water repellent portion 115 or the heat absorbing structure 116.

Although, in the above first and second embodiments, the first and second water repellent portions 115 and 122 and the heat absorbing structure 116 have frame shapes extending along the outer edges of the joint surfaces 114 and 121, the disclosure is not limited thereto. The first and second water repellent portions 115 and 122 and the heat absorbing structure 116 may be disposed only in a part of the outer edge.

Although, in the above second embodiment, the ultrasound transmitting and receiving portion 11 includes the heat absorbing structure 116, the disclosure is not limited thereto. The acoustic lens 12 may include the heat absorbing structure 116. Alternatively, as with the first and second water repellent portions 115 and 122 described in the above first embodiment, both the ultrasound transmitting and receiving portion 11 and the acoustic lens 12 may include the heat absorbing structure 116.

Note that the second embodiment includes the following technical ideas of appendant items 1 to 4.

1. An ultrasound probe including:

an ultrasound transmitting and receiving portion configured to transmit and receive ultrasound; and

an acoustic lens configured to radiate ultrasound emitted from the ultrasound transmitting and receiving portion to outside, in which

the ultrasound transmitting and receiving portion and the acoustic lens are joined to each other with an adhesive including a base resin and a curing agent, and

at least either a joint surface of the ultrasound transmitting and receiving portion or a joint surface of the acoustic lens, the joint surfaces being joined to each other with the adhesive, includes a heat absorbing structure configured to absorb heat of the adhesive on an outer edge side of the joint surface.

2. The ultrasound probe according to appendant item 1, in which

the ultrasound transmitting and receiving portion includes

a transducer including a plurality of piezoelectric elements, each of the piezoelectric elements being configured to emit ultrasound in response to an input of an electrical signal and convert ultrasound incident from outside to an electrical signal, and

an acoustic matching layer staked on the transducer and configured to perform acoustic impedance matching between the transducer and an object to be observed,

the acoustic matching layer and the acoustic lens are joined to each other with the adhesive, and

the heat absorbing structure is disposed on the outer edge side of at least either the joint surface of the acoustic matching layer or the joint surface of the acoustic lens, the joint surfaces being joined to each other with the adhesive.

3. The ultrasound probe according to appendant item 1 or 2, in which

the heat absorbing structure is

formed in a frame shape extending along an outer edge of the joint surface.

4. The ultrasound probe according to any one of appendant items 1 to 3, in which

the heat absorbing structure is

disposed outside an ultrasound transmitting and receiving area where the ultrasound transmitting and receiving portion transmits and receives ultrasound on a stacked cross-section of the ultrasound transmitting and receiving portion and the acoustic lens.

The ultrasound probe according to the disclosure achieves the effect of appropriately checking whether the adhesive inside has been cured.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound probe comprising:
an ultrasound transmitting and receiving portion configured to transmit and receive ultrasound; and
an acoustic lens configured to radiate ultrasound emitted from the ultrasound transmitting and receiving portion to outside and transmit ultrasound incident from outside to the ultrasound transmitting and receiving portion, wherein
the ultrasound transmitting and receiving portion and the acoustic lens each include a joint surface and a water repellent portion having water repellency disposed on an outer edge side of the joint surface, the joint surface of the ultrasound transmitting and receiving portion and the joint surface of the acoustic lens being joined to each other with an adhesive including a base resin and a curing agent.

2. The ultrasound probe according to claim 1, wherein
the ultrasound transmitting and receiving portion includes
a transducer including a plurality of piezoelectric elements, each of the piezoelectric elements being configured to emit ultrasound in response to an input of an electrical signal and convert ultrasound incident from outside to an electrical signal, and
an acoustic matching layer staked on the transducer and configured to perform acoustic impedance matching between the transducer and an object to be observed,
the acoustic matching layer and the acoustic lens are joined to each other with the adhesive,
the water repellent portion of the ultrasound transmitting and receiving portion is disposed on an outer edge side of a joint surface of the acoustic matching layer to which the joint surface of the acoustic lens is joined with the adhesive, and
the water repellent portion of the acoustic lens is disposed on an outer edge side of the joint surface of the acoustic lens to which the joint surface of the acoustic matching layer is joined with the adhesive.

3. The ultrasound probe according to claim 1, wherein
the water repellent portion is formed in a frame shape extending along an outer edge of the corresponding joint surface.

4. The ultrasound probe according to claim 1, wherein
the water repellent portion is disposed outside an ultrasound transmitting and receiving area where the ultrasound transmitting and receiving portion transmits and receives ultrasound on a stacked cross-section of the ultrasound transmitting and receiving portion and the acoustic lens.

5. The ultrasound probe according to claim 1, wherein
the water repellent portion includes a water repellent coating film disposed on the corresponding joint surface.

6. The ultrasound probe according to claim 1, wherein
the water repellent coating film is a fluorine coating film or a hydrophobic silica coating film.

7. The ultrasound probe according to claim 1, wherein
the water repellent portion has a double roughness structure including a plurality of micrometer-size projections including nanometer-size projections.
